(19) United States
(12) Patent Application Publication
(10) Pub. No.: US 2006/0241754 A1
(43) Pub. Date: Oct. 26, 2006

(54) Implant filling material and method

(76) Inventors: Arthur A. Beisang, White Bear Lake, MN (US); Robert A. Ersek, Austin, TX (US); Arthur A. Beisang III, North Oaks, MN (US); Daniel J. Beisang, North Oaks, MN (US)

Correspondence Address: NIKOLAI & MERSEREAU, P.A., 900 SECOND AVENUE SOUTH, SUITE 820, MINNEAPOLIS, MN, 55402, US

(21) Appl. No.: 11/450,644

(22) Filed: Jun. 9, 2006

Related U.S. Application Data
(63) Continuation-in-part of application No. 10/839,559, filed on May 5, 2004.
(60) Provisional application No. 60/533,168, filed on Dec. 30, 2003.

(51) Int. Cl. A61F 2/12

(52) U.S. Cl. 623/8

(57) ABSTRACT

Compositions of heat treated, cross-linked polyvinylpyrrolidone (PVP) are disclosed that are generally in the form of an elastic, hydrophilic, water insoluble viscous cohesive mass of material that has many important medical uses including uses as a filler for implants. The present invention also involves a process for producing such compositions.

The present application is a Continuation-In-Part of co-pending application Ser. No. 10/839,559, filed on May 5, 2004, the entirety of which is incorporated herein by reference for any purpose. That application, in turn, claims priority based on provisional application No. 60/533,168, filed Dec. 30, 2003, which is hereby also incorporated herein by reference in its entirety.

### BACKGROUND OF THE INVENTION

I. Field of the Invention

The present invention relates generally to medical implants and, more particularly, to implantable prostheses and materials used for same. The invention also relates to a process for making such materials.

II. Related Art

Medically implantable prostheses, exemplified by breast implants, are well known in the art. Such implants generally comprise a formed body presenting a nonreactive, biocompatible outer surface to surrounding tissue following implantation. Fluid-filled medical implants generally comprise a viscous fluid contained within an elastomeric shell. It has been observed that fluid-filled medical implants may leak or rupture following implantation and require explanation. The escaping fluid filler material may be contained within a periprosthetic capsule that forms around the prostheses after implantation, or it may be released into the body. It would present a desirable advantage to provide a filler for an implantable soft tissue prosthesis wherein the filler itself is substantially cohesive to facilitate removal of the filler from the body in the event of a rupture. It would present an additional advantage were this material composition nontoxic and preferably bioabsorbable.

Filling materials disclosed in previous patents relating to breast implants containing the polymer polyvinylpyrrolidone (PVP) have had some drawbacks that have been demonstrated and reported in the cosmetic plastic surgery literature. These implants generally are constructed with a silicone membrane shell. These drawbacks relate to the osmotic pressure created within the silicone shell membrane of the breast implant by the PVP solutions that have been previously utilized as filling material in clinical settings. The drawbacks also relate to control of the viscosity, cohesiveness, and elasticity of the PVP mixture used for filling breast implants.

Cross-linked PVP has a history of patented processes for the preparation of cross-linked PVP products, now commercially available from two major corporations: ISP and BASF. Three such patents are U.S. Pat. Nos. 2,938,017, 3,759,880, and 3,933,766. In the previous literature describing the process for obtaining cross-linked PVP, the temperature at which the cross-linking of PVP occurred has been required to be 100° C. or higher. Known processes for cross-linking PVP have required compounds or conditions which make them difficult to control. The rapid rate of cross-linking PVP in the aforementioned patents prohibits precise control of the cross-linked PVP products. Prior processes including that described in U.S. Pat. No. 3,933,766 call for the use of a cross-linking compound such as a cyclic acid amide or alkoxides in high pH environments (10-12 pH) or special commercial chemical “cross-linkers” at temperatures of 150° C. and pressures of 100 mm Hg.

Tacky, hydrophilic gel dressings have been disclosed using poly(N-vinyl lactam)-urethane gels in which the poly (N-vinyl lactam) may be polyvinylpyrrolidone (PVP) in U.S. Pat. Nos. 5,156,601 and 5,258,421. A skin adhesive hydrogel formed by mixing high molecular weight PVP having ring opened pyrrolidone groups and a multi-functional amine-containing polymer is disclosed in U.S. Pat. No. 5,306,504.

A further patent to Hayes et al, U.S. Pat. No. 5,997,574, discloses a medical implant which includes a shell and a filler material in which the filler material requires a combination of materials, namely, both a rheological agent and an osmotic control agent that is different from the rheological agent. In particular, Hayes et al teaches the use of polyvinylpyrrolidone (PVP) as an osmotic control agent in combination with a gum, preferably guar gum, as the rheological control agent. The combination is considered essential in order to provide a filler material having the desired properties for a breast implant, for example. The filler material of Hayes et al, further, is a one-phase, water-soluble mixture.

A long-standing need in the art for an improved formulation of PVP mixture for a filling material in breast implants has been recognized by the inventors and a new formula has been compounded and proposed. Accordingly, one aspect of the present invention relates to an improvement in cohesiveness, osmolarity, elasticity, and the viscosity of cross-linked PVP mixtures and mixtures of cross-linked PVP derivatives so that they can be controlled to be more favorable as filling material for breast implants and other uses. The proposed material composition is a viscous, highly elastic and cohesive mass comprised primarily in one embodiment of a lattice of water insoluble, heat-treated, cross-linked PVP and water.

### SUMMARY OF THE INVENTION

According to the present invention, there is provided a composition of heat-treated, cross-linked PVP that is in the form of an elastic, hydrophilic, water insoluble viscous cohesive mass of material that has many important medical uses. The present invention also involves a process for forming such a composition.

By way of definition, as used herein, the term “cohesive mass” refers to a unitary body composed of a pliant material, wherein when the unitary body is subjected to an external force directed toward disrupting the cohesive structural integrity of the unitary body, the unitary body resists fragmentation and retains its structural integrity.

Thus, the cohesive mass is a material consisting essentially of an aqueous solution of a single polymeric agent and a base and is preferably in the form of a hydrogel, or the like, that possesses generally gel-like elastic properties. Hydrogels, or water-containing gels, are defined as polymers characterized by hydrophilicity and insolubility in water. In water, they swell to an equilibrium volume, but preserve their shape, the hydrophilicity being due to the presence of water-solubilizing groups and the stability of the shape due to the presence of a three-dimensional network. Most preferably, the compositions of the cohesive mass of the present invention consist essentially of materials which are insoluble in water, but which are able to imbibe water without loss of shape or mechanical strength. They further do not require additional rheological agents such as gums to operate successfully as filler materials for medical implants. Thus, the filler materials of the present invention may also be said to be free of separate rheological control agents that characterize certain earlier filler materials. Smaller trace amounts of other materials may be present after heat treatment which do not affect the essential properties of the stable gel-like mass. Thus, the cohesive mass may be said to consist essentially of a hydrogel.

Also, the composition may be referred to as cross-linked PVP and may include chemically modified PVP or PVP derivatives which result from the process of the invention.

The process of the invention involves a heat-treatment step and a single polymeric agent in which a mixture consisting essentially of a water solution of PVP and a minor amount of a basic material such as sodium bicarbonate (NaHCO₃), for example, are heated at a temperature approaching, but remaining below the boiling point of water, i.e., just below 100° C., at a pressure of about 1 atm., for a sufficient time to produce the desired amount of reaction in the PVP. This results in a cross-linked or otherwise modified PVP polymer mass which can be described as cross-linked PVP or a modified PVP or a PVP derivative as the exact chemical structure is not known. The viscosity of fully processed material has been observed to be about 45,000 centipoise. One material was observed to be a 42% lattice of water-insoluble, heat-treated, cross-linked PVP and water.

In accordance with the process of the invention, it has been found that a solution containing K-30 PVP, for example, and an amount of sodium bicarbonate which is about 1% of the weight of the PVP, which is held for about 80 hours at 98° C., will produce a viable cohesive mass suitable for implants closely mimicking breast or other bodily tissue. The reaction can be carried on in the actual shell of use or in a shaped vessel shell that can later be dissolved away, if desired. The process also includes steps for the removal of unwanted high molecular weight fractions of PVP prior to heat treatment. The processing temperatures of the present invention enable the heat-treated material to maintain its initial volume. This is an important factor in the manufacture of implant products.

It has been found that various embodiments of the cross-linked PVP or PVP derivative, which makes up the cohesive mass, have a variety of additional medical uses. In this regard, for example, the material in sheet form can be utilized to prevent adhesions after surgical procedures by placing a sheet between the organs and the incision site. This property may be enhanced by removing additional water from sheets of the cohesive mass composition by heating or freeze drying. The material is also capable of being injected by bolus injection as a filler for tissue or as a carrier for drugs or other therapeutic agents in treatment of human subjects.

### BRIEF DESCRIPTION OF THE DRAWING

FIG. 1 represents a curve of viscosity of processed PVP material versus time as an illustration of the general process results.

### DETAILED DESCRIPTION

As indicated, previous literature demonstrates that the cross-linking of PVP must take place in the presence of cross-linking agents or compounds such as cyclic acid amides; and previous literature indicates that the cross-linking of PVP must take place at temperatures of 100° C. or higher. Prior formulations of PVP incorporate between 4% and 25% by weight of PVP in water, and the swellable gels which can be formed in these processes do not have viscocities approaching even 15,000 cp. It was, therefore, unexpected that the present invention could result in a highly elastic, extremely viscous, hydrophilic, swellable, cohesive mass with controlled rates of the cross-linking or other reaction occurring at between 37° C. and 100° C. This occurs without any commercial “cross-linker” and only 0.427% by weight of biocompatible sodium bicarbonate added to the PVP-initial water solution; and the PVP-water solution is about 42% PVP by weight. It was also surprising to discover that, in the method of the present invention, the cross-linking process can be stopped at any point by lowering the temperature to 25° C. The resulting product can include an insoluble PVP lattice and soluble PVP which requires filtration to separate the two phases. Centrifugation surprisingly, and unlike other processed cross-linked PVP does not separate the cross-linked PVP lattice from the soluble PVP. It is also surprising, in view of the previous literature, that reaction in the present invention occurs in the formulation at a neutral or slightly acid pH level of 6.5 (+/0.6) and lower.

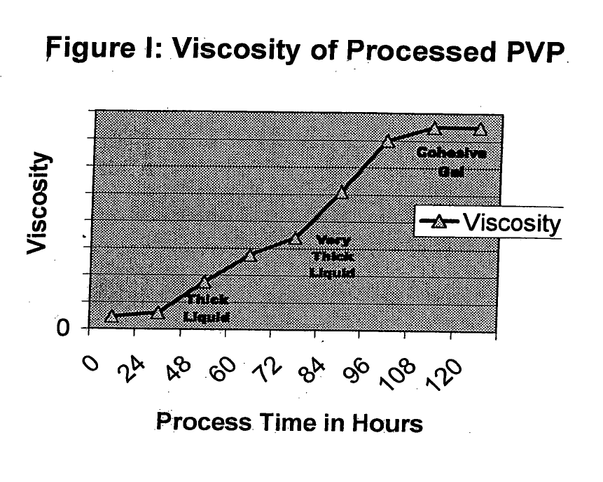

The above formulation for the cross-linked PVP mixture of the present invention has been physically mixed and compounded and measurements of essential physical characteristics such as viscosity, pH, weight, and freezing point depression of the invented mixture have been recorded. Implants containing the new formulation of cross-linked PVP were found to be stable after being treated and tested by placing them in a normal saline bath at body temperature (37° C.), and at 1½ times body temperature (55° C.) and at room temperature (25° C.) for a period of sixteen months. The new formulation was designed to improve the viscosity, cohesiveness, elasticity, and eliminate any problem that might occur from hypertonic formulations that had been previously recorded in the literature as increasing the volume and weight of implanted breast implants. For example “Long Term Results of MISTI Gold Breast Implants: A Retrospective Study”, Hildegunde Piza-katzer, M D., et. al., Plastic and Reconstructive Surgery, November 2002. The formula of the present invention has resulted in forming a highly viscous very cohesive and elastic mass of cross-linked PVP when treated for the appropriate length of time (see FIG. 1) at a temperature not exceeding that of boiling water (100° C.). The data for the graph of FIG. 1, for example, was acquired at a reaction temperature of 95° C.

In conjunction with the present invention, it should be noted that the precise chemical reactions that occur during the processing of the PVP in accordance with the process of the present invention are somewhat unclear. As far as it is presently understood, it is believed that the PVP polymer undergoes chemical changes during the heat treating step. These changes are believed to involve opening of the lactam ring that is part of the original PVP polymer, resulting in the formation of amino acid groups that are incorporated into the “modified” polymer. Thus, in this case, of course, the heat-treated polymer may no longer be a simple PVP structure. It may be a PVP that has been modified to create new chemical moieties incorporated into the polymer structure. Such modifications may likely be responsible for the ability of the material to possess its gel-like properties. This being the case, as indicated, references to cross-linked PVP herein, with respect to the materials produced by the present process are meant to include such modifications of PVP or derivatives of PVP as might occur as a result of the process of the invention.

According to an important aspect of the present invention, it has been found that a water mixture of PVP and sodium bicarbonate can be maintained at a constant volume and weight when it is heated at a temperature between normal body temperature and the boiling point of water so that the material will maintain its original volume and weight for as long as water is not lost from the system. The temperature at which the mixture is heated along with the length of time the mixture is heated at a given temperature precisely results in a viscosity that is controlled to be anywhere between the viscosity of the beginning mixture (approximately 1000 cp) to viscosity of the cross-linked PVP viscous cohesive mass (about 45,000 cp) (see FIG. 1). It has been determined that the viscosity of the initial formulation can be increased in a controllable manner by the treatment of heat over a period of time. The volume of the initial mixture is held constant in a controllable manner. The cohesiveness is increased in a controllable manner, and the osmotic pressure is eventually made irrelevant as the heat treatment viscosity and temperature is increased in a controlled manner.

It has been demonstrated, for example, that a viscosity greater than 15,000 centipoise and up to about 45,000 centipoise maximum cohesiveness and elasticity of the cross-linked PVP cohesive mass is achieved with no change in volume or weight of the mixture. It appears that the osmolarity is almost irrelevant at this level of viscosity and cohesiveness because the cross-linked insoluble PVP cohesive mass does not appear to have a significant osmotic pressure. Thus, the composition has essentially all of the PVP cross-linked or otherwise entrapped in the viscous cohesive mass structure which results in low or no osmotic pressure. In addition, PVP is hydrophilic; therefore the hydrophilic force may work to prevent water from moving out of the breast implant through the silicone membrane to dilute the higher osmotic pressure of the tissue fluids. This of course is a very significant factor and a great improvement in PVP filling materials for breast implants.

The formulation of water, sodium bicarbonate and PVP can be treated for a period of time at a temperature below boiling necessary to achieve viscosities greater than 15,000 centipoise and up to about 45,000 centipoise form a completely cohesive, water insoluble mass with no change in weight or volume. The cohesive mass that is formed contains substantially all of the initial water but the PVP is no longer soluble and is in the form of a viscous cohesive mass in which the insoluble PVP remains hydrophilic.

The above properties are all desirable characteristics for a breast implant filling material and such a filling material will eliminate several negative aspects of filling materials previously used. Sodium hydroxide may be substituted for sodium bicarbonate in the initial mixture with the identical PVP cross-linked hydrogel being formed. It is contemplated that other ionic molecules may also occur to those skilled in the art to be substituted for sodium bicarbonate. It will be appreciated that the general process for making the above-described cohesive mass has useful applications in various medical fields. Some of these will be described.

### First Embodiment

First, PVP and sodium bicarbonate are dissolved in water in a ratio recorded hereafter. The materials must be dissolved thoroughly and the mixture degassed, if needed. The mixture is then introduced into an implant shell, such as any silicone shell for a penile implant or breast implant or any kind of an anatomical implant that can be filled with this mixture. In the next step, the implant shell is heated containing the mixture at a temperature that remains below the boiling point of water and at a pressure of about 1 atm for a sufficient period of time to cause the amount of cross-linking of the PVP which is desired for that particular implant (see FIG. 1). As an example of this process one might dissolve 75 grams of K-30 PVP and 0.7 grams of sodium bicarbonate in 100 grams of water. A volume of that mixed fluid solution sufficient to fill a thin silicone implant shell is then introduced into such a shell. Next, that solution in the silicone container or shell is treated for a period of 80 hours at 98° C. The filling material will complete its cross-linking in this time at this temperature and will retain its initial weight, volume and shape as a viscous cohesive mass and upon sterilization will be ready for use as an anatomical implant.

### Second Embodiment

The previous embodiment of the process requires a mold or container and pre-supposes that the mold or container for the gel-like cohesive mass which will be formed will be implanted as part of the anatomical implant, breast implant or penile implant. This, however, is optional and so is not a necessary characteristic of the process of the invention. The formulation of water, PVP and sodium bicarbonate as mixed can be optionally introduced into a mold other than a mold that will be utilized as a membrane container for the implant. As an example of an alternate mold, one should be able to make a mold of sodium bicarbonate crystals, introduce the fluid and surround the fluid by the sodium bicarbonate mold and then proceed with the process of heating the fluid in the mold at a temperature and pressure less than that required to reach the boiling point of water. This would take approximately 80 hours at a temperature of 98° C. at atmospheric pressure. The desired cohesive mass formed by cross-linking PVP molecules retains almost all of the original water of the formulation. The mold can be released by physical means, or in the case of sodium bicarbonate, the mold can be dissolved away with water and the insoluble cohesive mass composition could be retrieved. The water solution used to dissolve the sodium bicarbonate would not dissolve the cross-linked PVP and the remaining material retrieved and recovered would be the cohesive mass of insoluble cross-linked PVP in the molded shape that was desired.

It is also anticipated that other ionic molecules such as persulfate or sodium acetate may be used in the initial mixture as an alternative to sodium bicarbonate, but present experiments have involved sodium bicarbonate and sodium hydroxide.

### Third Embodiment

This aspect involves the separation and removal of a high molecular weight fraction that is undesirable in the human body. Commercial PVP products available from the manufacturers (BASF or ISP), K-15 and K-30 generally contain between 2-14% by weight of a high molecular weight fraction of PVP molecules (defined as molecules having a molecular weight over 100,000). This fraction is an undesirable feature of PVP that is soluble in a water solution because it has been reported that human kidneys do not remove PVP of molecular weight 100,000 or higher and, consequently, such PVP remains in tissues rather than being eliminated through urine. As stated above, the present invention also deals with the separation and removal of this high molecular weight fraction prior to the making of the gel-like cohesive mass implant products of the invention. This aspect of the process, as in the examples above, starts with the fluid containing commercially obtained PVP, water and sodium bicarbonate. The fluid material is heated for an appropriate time to enable approximately 10-20% of the PVP by weight to become cross-linked, thereby increasing the viscosity to approximately 3000 centipoise. Because the large molecular weight molecules of PVP are the first to cross-link, the resultant insoluble PVP of the solution will be comprised of the fraction including the larger molecular weight molecules that have cross-linked and the remainder of PVP molecules (molecular weight <100,000) will remain soluble in the mixture. The two PVP materials can be separated by filtration or other physical means, resulting in a process solution of water-soluble, lower molecular weight PVP molecules (molecular weight less than 100,000).

For example, a solution of 750 grams of PVP in 1000 grams of water with 7.5 grams of biocompatible ionic compound such as sodium bicarbonate added would be thoroughly mixed and degassed. Thereafter, that mixture would be treated with heat at 98° C. for a period of 29 hours or another suitable time. The cross-linking that would occur during that period of time would involve approximately 20% of the original weight of PVP. The mixture of soluble PVP and cross-linked insoluble PVP could then be separated by filtration. Standard gel permeation analysis of the PVP portion that is soluble in water of separated material will demonstrate that there are no PVP molecules in the remaining soluble fraction having a molecular weight greater than 100,000. The remaining soluble fraction can be used to make acceptable gel-like products as above by using salt/heat to get to 15000 cp to 45,000 cp, as previously detailed.

### Fourth Embodiment

In the previous examples, the viscous cohesive mass of water insoluble, cross-linked PVP material made according to the process maintained its initial volume. In some cases for medical use it is desirable to create a scaffolding of PVP, which maintains its original shape but contains little water. It has been found that a modified sheet form of the viscous cohesive mass of cross-linked insoluble PVP gel-like material of the present invention can be utilized to prevent adhesions after a surgical procedure by placing the sheet between the organs and an incision site (see the Second Embodiment). In addition, in another field of medicine, it would be useful to have a PVP sheet similar to that described, with additional water removed from the sheet to provide a scaffold that would induce and be a suitable scaffold to promote cellular growth either outside or inside the body, in vitro or in vivo.

Accordingly, it is a further aspect of the present invention to provide a procedure that will produce such a sheet structure that is substantially devoid of water. In this case, an appropriate volume of PVP such as K-30 PVP, water, and appropriate weight of sodium bicarbonate, as in the above examples, would be mixed thoroughly and degassed. The fluid mixture would be poured into a container to form a sheet of the desired thickness, which might be approximately one tenth of an inch, or one quarter of an inch, or even one half of a inch thick. The poured mixture would then be heated to 98° C. for approximately 82 hours. At this time a viscous cohesive mass of PVP, insoluble in water, would have formed. The sheet would then be freeze-dried by placing the sheet at a freezing temperature in a vacuum chamber and maintaining those conditions until essentially all the water was removed from the sheet in a well-known manner. Alternatively, substantially all of the water could be removed or driven off from the cohesive mass composition sheet by increasing the temperature (heating in an oven).

Appropriate portions of the freeze-dried sheet in combination with the proper nutrients can be used in vitro or in vivo to grow various mammalian cells. Freeze-drying the cohesive mass in sheet form will maintain and preserve the initial shape of the sheet. If the viscous cohesive mass in sheet form is not freeze-dried, but heat is continued to be applied to the viscous cohesive, insoluble PVP sheet, the trapped water will be driven off and the sheet will become a hard or solid sheet of 20-40 percent of the initial volume of the hydrated gel-like sheet. This method for making solid articles that have more solid properties by driving off the water with vacuum or heat also can be employed when a more solid implant material is desirable.

### Fifth Embodiment

For some medical uses it is desirable to have a material such as the PVP described previously injected as filler for tissue. For instance, in the field of urology, the viscous cohesive mass made by the process of the present invention may be used as a bulking material that is introduced to the inside of a sphincter muscle by being injected from a syringe. Such a bulking material is also used for filling tissue in scars, dents of the skin, and for remodeling chins, noses, lips, ears, etc. For this procedure, a formulation of soluble PVP, water and sodium bicarbonate can be introduced into syringes of 1, 2, and 3 cc volumes, for example. The formulated fluid of PVP would be cross-linked inside the syringes. They would be placed in an environment where the syringes could be heated to a temperature less than that of boiling water for a length of time sufficient to enable the desired amount of cross-linking of PVP to occur within the syringe to attain an appropriate viscosity for injection in soft tissues. Thus, if the syringe were heated at 98° C. for a period of 90 hours, for example, a viscosity consistent with that of other materials (injectable bulking materials) would be achieved. This would be a viscosity in excess of 15,000 cp. Depending on the formulation and conditions under which the cohesive mass is processed and injected, the cohesive cross-linked mass should remain at the site of injection for an indeterminate period, possibly providing permanent or semi-permanent bulking. If small particles of synthetic biocompatible material such as dimethylsiloxane are added to the material, the particles will remain as a permanent bulking agent.

### Sixth Embodiment

The object of this novel embodiment is to increase the surface area of the volume of cross-linked PVP material. The first step is to form a substantial volume of the viscous cohesive mass of cross-linked PVP composition such as was described in the Second Process Embodiment. A volume of water is added to the volume of viscous cohesive mass of material in a desired selected ratio, such as 1:1. The cohesive mass, which is insoluble in water, is then mechanically broken into pieces using a vigorous, mechanical disruption means such as a blender, for example. Because each of the small pieces created is hydrophilic on its entire surface, the mixture will become as a fluid that can be used to coat tissue surfaces such as internal organs. In addition to the sheet forms indicated above, such a fluid can be used to coat tissue and organ surfaces during and after surgical procedures to form a barrier that reduces unwanted adhesions between the operative site and internal tissue and organs. This fluid can be administered as a spray or as an injectable depending upon the needs of the surgeon to prevent adhesions.

### Seventh Embodiment

This aspect or embodiment of the process of the invention makes use of the higher molecular weight fraction separated from commercial PVP as described above or uses higher molecular weight commercial material. As indicated, the PVP selected is comprised of large molecules, in excess of 100,000 molecular weight. For example, K-60 or K-90 PVP is dissolved in a 0.42% by weight sodium bicarbonate solution and heated at 98° C. for a period of 60 hours to produce a viscous cohesive mass of cross-linked PVP having a viscosity in excess of 15,000 cp up to about 45,000 cp. This hydrophilic material, while highly viscous, can be introduced into a joint using a syringe and needle delivery system. The material is also lubricious and, in this manner, will serve as a lubricant for joint surfaces.

### Eighth Embodiment

It is contemplated that the cross-linked PVP material made by the process of the present invention can advantageously be employed for still other medical uses. For example, for some medical uses it would be desirable to have a material such as the cross-linked PVP described in relation to the process of the Second Embodiment, for example, that can be used as a drug delivery system. For instance a drug may advantageously be mixed with a substantial volume of the viscous cohesive mass of cross-linked PVP material such as described in the Second Embodiment. The gel-like material and drug mixture can then be placed in contact with the body as through injection as a bolus, transdermally through contact with the skin or by other well known means. Typically, the drug will migrate out of the cohesive mass and be made available to the body. For example, a PVP viscous cohesive mass was prepared as in the Second Embodiment process and a red, water-soluble, food dye was incorporated into the formulation. The now-colored gel-like insoluble material was placed into a container containing water and allowed to remain in contact with the water. After time, the water became colored red, indicating transfer of dye from the insoluble cohesive mass into the water phase. In another example, a PVP material was prepared using the process of the Second Embodiment and placed into a container containing a water solution of a red, water-soluble, food dye. After time, the bolus of the insoluble composition was removed from the water solution and observed. Quantities of the red dye had migrated into the insoluble cohesive mass. These examples demonstrate that pharmacologically active materials could be delivered to the body via migration into or out of the cohesive mass of cross-linked PVP produced in accordance with the invention.

The curve of the graphical representation shown in FIG. 1 was derived according to the procedure next described. A water solution comprised of 43% by weight polyvinylpyrrolidone and 0.4% by weight NaHCO₃ was heated at 95° C. and a record of the viscosity was measured over a period of 108 hours. A logistic regression was performed on the experimental viscosity over time data. This regression resulted in the following equation:
V(t)=A/(1+Be^{ct})+D.
Where:

D is a vertical shift to correct for the initial viscosity,

C is an empirically derived growth factor which is affected by temperature and catalyst, and

B and A are constants having to do with the initial viscosity and the final asymptotic viscosity.

“t” is the time variable in which the units are hours and the function V(t) gives the viscosity at time “t” in centipoises.

For this experiment, our values were as follows: A=41254; B=305.09; C=0.074753; D=997.7; e=mathematical constant.

The production of a cross-linked PVP material, which is not soluble in water, per se, is well known and has been reported previously. The procedures reported previously, however, have all involved reacting soluble PVP in a highly alkaline environment and/or employing commercial cross linkers, which formed cross-linked molecules at temperatures in excess of 100° C.

In contrast, all the processes of the present invention involve temperatures less than 100° C. or at least at temperatures and pressures that are low enough to prevent the water in the original formulation from boiling. In addition, it is noteworthy that the measured pH of the initial formulation of the present invention prior to cross-linking is 6.4+/0.6. Thus, the procedure of the present invention allows the formulation of insoluble PVP in volumes and shapes that are desirable for various medical uses. The invented procedure also allows water soluble PVP to be produced where the molecular weight can be controlled to be within a desirable range, such as the range of molecular weights below 100,000, so that the condition of tissue storage may be eliminated. The process of the invention also allows the insoluble PVP to remain a specific volume and shape and form a cohesive viscous mass of material which has not been possible in previous processes that requires processing temperatures above 100° C. At processing temperatures above 100° C. water is lost and the volume is reduced, therefore the shape and volume is reduced and unpredictable.

The cohesive mass may be produced in a form that can be injected to augment tissue, in addition to a form that can be used as a filling material in anatomical implants. The composition of the present invention may be used in many other applications that require a cohesive gel.

The combination of water insoluble cohesive gel mixed with PVP soluble in water can be used as an injectable material. Because PVP in the soluble form or in the water insoluble form is biocompatible and does not illicit a immunological allergenic reaction in the body, the medical devices described previously can be comprised of PVP that has been treated in the manor described to become water insoluble in the form of a cohesive water insoluble composition, or the cohesive water insoluble PVP material can be used in combination with PVP water soluble fluid. A combination of water-soluble and water insoluble PVP can be used for a biocompatible anti-adhesion composition and in various other medical devices.

Viscosities of the processed cohesive mass material may range from a low of about 10,000 cp to about 45,000 cp or more. A preferred range starts at 15,000 cp or above.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. An implantable prosthetic body made by a process including the steps of:
(a) presenting a thin elastomeric container;
(b) filling said elastomeric container with a filler material consisting essentially of an aqueous solution of a single polymeric agent and a base, wherein said single polymeric agent is polyvinylpyrrolidone and said filler material has a viscosity less than 10,000 centipoise; and
(c) heating said filler material at a temperature less than 100° C. until said filler material has a viscosity greater than 10,000 centipoise.

2. An implantable prosthetic body of claim 1 wherein said prosthetic body has the general shape of a breast.

3. An implantable prosthetic body of claim 1 wherein said prosthetic body has the general consistency of breast tissue.

4. An implantable prosthetic body of claim 1 wherein said base is a metal hydroxide.

5. An implantable prosthetic body of claim 1 wherein said base is NaHCO₃.

6. An implantable prosthetic body of claim 1 wherein said base is NaOH.

7. An implantable prosthetic body of claim 1 wherein the pH of the filler material is less than 7.0.

8. An implantable prosthetic body comprising:
(a) a thin elastomeric container; and
(b) a pliable viscous cohesive mass consisting essentially of a hydrogel contained within said container.

9. An implantable prosthetic body as in claim 8 wherein said cohesive mass has a viscosity greater than 10,000 centipoise.

10. An implantable prosthetic body as in claim 8 wherein said pliable viscous cohesive mass comprises at least 40% (w/v) polyvinylpyrrolidone or a derivative thereof.

11. An implantable prosthetic body as in claim 9 wherein said pliable viscous cohesive mass comprises at least 40% (w/v) polyvinylpyrrolidone or a derivative thereof.

12. An implantable prosthetic body as in claim 10 wherein said prosthetic body has the general shape of a breast.

13. An implantable prosthetic body as in claim 10 wherein said prosthetic body has the general consistency of breast tissue.

14. An implantable prosthetic body as in claim 11 wherein said prosthetic body has the general shape of a breast.

15. An implantable prosthetic body as in claim 11 wherein said prosthetic body has the general consistency of breast tissue.

16. An implantable prosthetic body as in claim 9 wherein said cohesive mass has a viscosity greater than 15,000 centipoise.

17. An implantable prosthetic body consisting of a pliable viscous cohesive mass comprising polyvinylpyrrolidone or a derivative thereof, said cohesive mass having a viscosity greater than 10,000 centipoise.

18. The implantable prosthetic body of claim 17 wherein said prosthetic body has the general shape of a breast.

19. The implantable prosthetic body of claim 17 wherein said prosthetic body has the general consistency of breast tissue.

20. A method of processing polyvinylpyrrolidone for separating a high molecular weight (molecular weight >100,000) fraction from lower molecular weight fractions comprising steps of:
(a) heat treating an initial mixture of commercially available, water soluble polyvinylpyrrolidone (PVP) having a range of molecular weights in the presence of water and a minor amount of a base at a temperature below 100° C. for a sufficient time to enable a selected fraction of the PVP to react and become insoluble in water; and
(b) physically separating the water insoluble fraction, allowing the remaining water soluble fraction to remain in solution.

21. A method as in claim 20 wherein said insoluble fraction is physically separated by means of filtration.

22. A method as in claim 20 wherein said heat treating step continues until the viscosity of the reacting material reaches 3,000 cp.

23. A method as in claim 20 wherein said heat treating step is carried out until an amount of polyvinylpyrrolidone greater than the known amount of the high molecular weight fraction in the original solution is reacted until it becomes insoluble in water.

24. A method as in claim 20 wherein said base is sodium bicarbonate.

25. A method of producing a pliable viscous cohesive mass comprising at least 40% (w/v) polyvinylpyrrolidone or a derivative thereof comprising steps of:
(a) heat treating a mixture of commercially available, water soluble polyvinylpyrrolidone in the presence of water and a minor amount of a base at a temperature below 100° C., at a pressure of about 1 atm, for a sufficient time to enable a resulting viscous cohesive mass to form and reach a selected viscosity; and
(b) allowing said reacted cohesive mass to cool.

26. A method as in claim 25 wherein said mixture is introduced into an implant shell prior to said heat treating step.

27. A method as in claim 25 wherein said mixture is introduced into a soluble container prior to said heat treating step and including the further step of dissolving away said soluble container after said cooling step.

28. A method as in claim 25 wherein said heat treating step is carried on until the viscosity of said cohesive mass is >10,000 cp.

29. A method as in claim 25 wherein said heat treating step is carried on until the viscosity of said cohesive mass is >15,000 cp.

30. A method as in claim 25 wherein said heat treating step is carried on until the viscosity of said cohesive mass is about 45,000 cp.

31. A method as in claim 25 wherein said temperature of said heat treating step is from about 95° C. to about 98° C.

32. A method as in claim 25 wherein said base is sodium bicarbonate.

33. A method as in claim 25 wherein said sufficient time is between about 80 hours and about 120 hours.

34. A method as in claim 26 wherein said implant shell is for a breast implant.

35. A method as in claim 25 wherein said heat-treating step is carried out with said mixture and is retained in flat form to produce a sheet of desired thickness of said cohesive mass.

36. A method as in claim 35 including the step of removing a desired amount of water from said sheet form of said cohesive mass by a method selected from the group consisting of freeze drying and heating.

37. A method as in claim 25 wherein said heat treating step is conducted with said mixture in a syringe.

38. A method as in claim 25 including the additional step of infusing an amount of a therapeutically active material into said cohesive mass.

39. A method as in claim 24 wherein the amount of said sodium bicarbonate in said initial mixture is about 1% of said PVP (w/w).

40. A method as in claim 32 wherein the amount of said sodium bicarbonate in said initial mixture is about 1% of said PVP (w/w).

41. A method as in claim 25 wherein the pH of said mixture is about 6.4+/0.6 during heat treating.

42. A method as in claim 32 wherein said temperature of said heat-treating step is from about 95° C. to about 98° C.

43. A method as in claim 42 wherein the amount of said sodium bicarbonate in said initial mixture is about 1% of said PVP (w/w).
